(19) United States
(12) Patent Application Publication
(10) Pub. No.: US 2026/0086007 A1
(43) Pub. Date: Mar. 26, 2026

(54) TRIAXIAL TEST DEVICE FOR DEEP-SEA CORING RETAINING IN-SITU CONDITION SUITABLE FOR SHIPBORNE LABORATORY

(71) Applicants: ZHEJIANG UNIVERSITY, Hangzhou, Zhejiang (CN); DONGHAI LABORATORY, Zhoushan, Zhejiang (CN)

(72) Inventors: Jiawang CHEN, Zhoushan, Zhejiang (CN); Zhangyong JIN, Zhoushan, Zhejiang (CN); Qiaoling GAO, Zhoushan, Zhejiang (CN); Han GE, Zhoushan, Zhejiang (CN); Zhonghui ZHOU, Zhoushan, Zhejiang (CN); Peng ZHOU, Zhoushan, Zhejiang (CN); Liwen NAN, Zhoushan, Zhejiang (CN)

(21) Appl. No.: 18/865,890

(22) PCT Filed: Sep. 14, 2024
(86) PCT No.: PCT/CN2024/118966

§ 371 Date: Nov. 14, 2024

(30) Foreign Application Priority Data
Nov. 20, 2023 (CN) 202311539966.8

(51) Int. Cl. G01N 3/08; G01N 3/02; G01N 33/24

(52) U.S. Cl. CPC G01N 3/08; G01N 3/02; G01N 33/24; G01N 2203/0234

(57) ABSTRACT

A triaxial test device for deep-sea coring retaining in-situ condition suitable for a shipborne laboratory, includes a sample transfer system and a triaxial machine system. The sample transfer system includes a sample cylinder, a tube-removing piston, a ball valve, and a connector, the sample cylinder is connected to the ball valve through bolts, the sample is placed in the sample cylinder, and the tube-removing piston arranged in the sample cylinder is configured to push the sample to enter the triaxial machine system. The triaxial machine system includes a bottom interface, a rubber cylinder, a triaxial inner wall, a triaxial outer wall, and a top interface; the bottom interface is hermetically connected to the sample cylinder through the connector, the bottom interface communicates with the rubber cylinder arranged in an inner cavity of the triaxial inner wall, the sample is sent into the rubber cylinder for a triaxial compression test.

### CROSS-REFERENCE TO RELATED APPLICATION

This patent application is a national stage application of International Patent Application No. PCT/CN2024/118966, filed on Sep. 14, 2024, which claims priority of Chinese Patent Application No. 202311539966.8, filed on Nov. 20, 2023, both of which are incorporated by references in their entireties.

### TECHNICAL FIELD

The present disclosure relates to a technical field of seabed core triaxial tests, and in particular to a triaxial test device for deep-sea coring retaining in-situ condition suitable for a shipborne laboratory.

### BACKGROUND

China is one of the largest energy producing countries and consuming countries in the world, and is rich in fossil energy. However, per-capita average of energy resources of China is low, and problems of energy safe supply and environmental pollution are getting worse, so there is an urgent need to develop environment-friendly energy products. Natural gas hydrate, as new clean energy, has characteristics of wide distribution, deep burial, no pollution and high energy density, and thus has extremely broad development prospect. However, ecological and environmental problems caused by exploitation of deep-sea energy soil (seabed sediments containing natural gas hydrate) are difficult problems that all countries need to solve urgently. Therefore, studying mechanical properties of deep-sea energy soil gas-bearing reservoir is related to safe exploitation of deep-sea energy, and has important theoretical and guidance significance for the development of new energy sources.

Triaxial test is one of the most widely used method to study soil strength and deformation characteristics, and is a test method suitable for studying the mechanical characteristics of deep-sea energy soil reservoirs. However, the traditional triaxial test device is often only suitable for geotechnical samples with low pressure at room temperature, which cannot meet test conditions of the deep-sea energy soil reservoirs. In order to solve the problem above, a triaxial test device for deep-sea coring retaining in-situ condition suitable for a shipborne laboratory needs to be designed.

### SUMMARY

An objective of the present disclosure is to provide a triaxial test device for deep-sea coring retaining in-situ condition suitable for a shipborne laboratory, which is used to study mechanical properties of deep-sea energy soil reservoir, and can well solve problems of a triaxial test for the deep-sea energy soil reservoir, thus providing an effective safety design scheme for safe exploitation of deep-sea energy soil, and providing corresponding theoretical support for reducing environmental risk caused by energy exploitation.

To achieve the objective above, the present disclosure provides the following solutions: a triaxial test device for deep-sea coring retaining in-situ condition suitable for a shipborne laboratory includes:

a sample transfer system, configured to remove a natural gas hydrate sample from a tube and send the natural gas hydrate sample to a triaxial machine system, where the sample transfer system comprises a sample cylinder, a tube-removing piston, a ball valve, and a connector, the sample cylinder is connected to the ball valve through bolts, the sample is placed in the sample cylinder, and the tube-removing piston arranged in the sample cylinder is configured to push the sample to enter the triaxial machine system; and

a triaxial machine system, configured to complete a triaxial test of the natural gas hydrate sample in a vacuum condition, where the triaxial machine system comprises a bottom interface, a rubber cylinder, a triaxial inner wall, a triaxial outer wall, and a top interface; the bottom interface is hermetically connected to the sample cylinder through the connector, the bottom interface communicates with the rubber cylinder arranged in an inner cavity of the triaxial inner wall, the sample is sent into the rubber cylinder for a triaxial compression test, the triaxial inner wall is filled with seawater for guaranteeing a test pressure, the triaxial outer wall at the periphery of the triaxial inner wall is provided with a water inlet and a water outlet, and circulating cold water communicates with the water inlet and the water outlet on the triaxial outer wall.

Preferably, the connector is a hold hoop, and the bottom interface is hermetically connected to the sample cylinder through the hold hoop.

Preferably, a triangular groove symmetrical up and down is formed on a tail end of the tube-removing piston, and when the sample is stored in the sample cylinder, oil between the tube-removing piston and the sample cylinder flows out through the triangular groove in an axial direction.

Preferably, a plastic tube is attached to an outer side of the sample placed in the sample cylinder, and a diameter of the tube-removing piston is smaller than an inner diameter of the plastic tube.

Preferably, a diameter of the bottom interface is smaller than an outer diameter of the plastic tube, the tube-removing piston in the sample transfer system is able to push the sample to move forwards under an action of seawater until the sample is abutted against the bottom interface of the triaxial machine system, and the sample is separated from the plastic tube under pushing of the tube-removing piston to enter the triaxial machine system.

Preferably, each of the bottom interface and the top interface is provided with confining pressure holes, and the confining pressure holes on the bottom interface and the top interface are configured to provide a confining pressure for the sample with cooperation of an external pressure device.

Preferably, the top interface is provided with pore pressure holes, the pore pressure holes are configured for testing a pore pressure of the sample in during the triaxial test.

Preferably, the rubber cylinder is fixedly installed in an inner cavity of the triaxial inner wall through a fixing ring.

Compared with the prior art, the present disclosure obtains the following beneficial technical effects:

A triaxial test device for deep-sea coring retaining in-situ condition suitable for a shipborne laboratory provided by the present disclosure includes a sample transfer system, and a triaxial machine system. A sample conforming to a length requirement of the triaxial test is stored in the sample transfer system, and the ball valve is closed to preserve a high-pressure environment of the sample. A tube-removing piston is used to remove the tube from the sample and send the sample to a triaxial test system, and a triaxial test is carried out in a rubber cylinder, thus mechanical properties of a deep-sea energy soil reservoir sample are obtained. The triaxial test device for deep-sea coring retaining in-situ condition can be carried on a research vessel conveniently, and can in butt joint with an existing natural gas hydrate sample pressure-retaining transfer system, thus performing a fidelity triaxial test immediately after obtaining a gas hydrate sample, and achieving strong scientific research benefits.

### BRIEF DESCRIPTION OF THE DRAWINGS

To describe the technical solutions of the embodiments of the present disclosure or in the prior art more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without creative efforts.

FIG. 1 is a schematic diagram of an overall structure of a triaxial test device for deep-sea coring retaining in-situ condition suitable for a shipborne laboratory;

FIG. 2 is a structural diagram of a sample transfer system;

FIG. 3 is a schematic diagram of a triaxial machine system.

In the drawings: 1-sample transfer system; 1-1-sample cylinder; 1-2-tube-removing piston; 1-3-sample; 1-4-ball valve; 1-5-hold hoop; 2-triaxial machine system; 2-1-bottom interface; 2-2-rubber cylinder; 2-3-fixing ring; 2-4-triaxial inner wall; 2-5-triaxial outer wall; 2-6-top interface.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by those of ordinary skill in the art based on the embodiments of the present disclosure without creative efforts shall fall within the scope of protection of the present disclosure.

An objective of the present disclosure is to provide a triaxial test device for deep-sea coring retaining in-situ condition suitable for a shipborne laboratory, which is used to study mechanical properties of deep-sea energy soil reservoir, and can well solve problems of a triaxial test for the deep-sea energy soil reservoir, thus providing an effective safety design scheme for safe exploitation of deep-sea energy soil, and providing corresponding theoretical support for reducing environmental risk caused by energy exploitation.

In order to make the objectives, features and advantages of the present disclosure more clearly, the present disclosure is further described in detail below with reference to the embodiments.

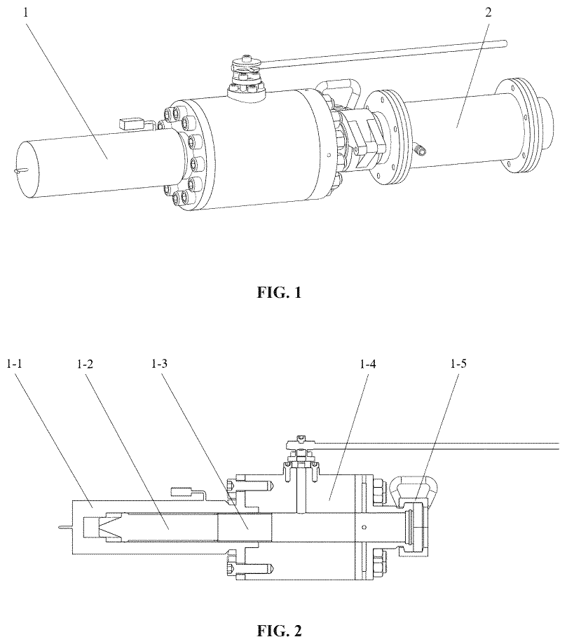

As shown in FIG. 1 to FIG. 3, a triaxial test device for deep-sea coring retaining in-situ condition suitable for a shipborne laboratory provided by the present disclosure includes a sample transfer system 1, and a triaxial machine system 2. The sample transfer system 1 is responsible for removing a natural gas hydrate sample from a tube and sending the natural gas hydrate sample to the triaxial machine system, and the triaxial machine system 2 is responsible for completing a triaxial test of the natural gas hydrate sample in a fidelity condition.

The sample transfer system 1 is responsible for removing the natural gas hydrate sample from the tube and sending the natural gas hydrate sample to the triaxial machine system 2. Firstly, the sample transfer system 1 is in butt joint with an existing natural gas hydrate sample pressure-retaining transfer system in market to store a fidelity sample with a cut length into a sample cylinder 1-1, and at this time, an outer side of the sample 1-3 is still has a plastic tube attached during sampling. Then a ball valve 1-4 in the sample transfer system 1 is closed, thus the sample transfer system 1 is in butt joint with the triaxial machine system 2 under an action of a hold hoop 1-5. Finally, a tube-removing piston 1-2 in the sample transfer system 1 pushes the natural gas hydrate sample 1-3 to move forward under an action of high-pressure seawater until the sample is abutted against a bottom interface 2-1 of the triaxial machine system 2. A diameter of the bottom interface 2-1 is designed to be slightly smaller than an outer diameter of the plastic tube, and a diameter of the tube-removing piston 1-2 is designed to be slightly smaller than an inner diameter of the plastic tube. The natural gas hydrate sample 1-3 is removed from the tube under the push of the tube-removing piston 1-2, and then enters the triaxial machine system 2.

As shown in FIG. 2, the sample transfer system 1 includes the sample cylinder 1-1, the tube-removing piston 1-2, the sample 1-3, the ball valve 1-4, and the hold hoop 1-5. The sample cylinder 1-1 is connected to the ball valve 1-4 throughbolts, and opening and closing of the ball valve 1-4 is used to preserve the high-pressure natural gas hydrate sample 1-3. The natural gas hydrate sample 1-3 is removed from the tube under the push of the tube-removing piston 1-2, and then enters the triaxial machine system 2. The hold hoop 1-5 is responsible for connecting the sample transfer system 1 to the triaxial machine system 2.

In one embodiment, a tail end of the tube-removing piston 1-2 is provided with a tapered structure to play a role of buffering. When the fidelity sample is stored in the sample cylinder 1-1 through the natural gas hydrate sample pressure-retaining transfer system, the oil between the tube-removing piston 1-2 and the sample cylinder 1-1 needs to flow out through the tapered structure in an axial direction, thus the tube-removing piston 1-2 is braked. A conical throttling area of this tapered structure decreases gradually with increase of a buffer stroke, thus making change of the buffer pressure evenly and reducing the impact pressure.

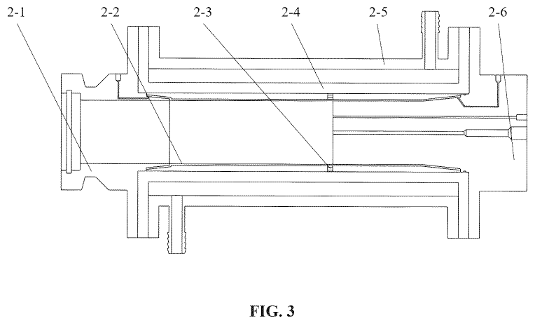

The triaxial machine system 2 is responsible for completing a triaxial test of the natural gas hydrate sample, and is composed of an inner layer and an outer layer. A triaxial inner wall is responsible for retaining deep-sea high pressure, and a triaxial outer wall is responsible for retaining deep-sea low temperature. Specifically, as shown in FIG. 3, the triaxial machine system 2 includes the bottom interface 2-1, a rubber cylinder 2-2, a fixing ring 2-3, the triaxial inner wall 2-4, the triaxial outer wall 2-5, and a top interface 2-6. Each of the bottom interface 2-1 and the top interface 2-6 is provided with confining pressure holes, which can provide a confining pressure for the natural gas hydrate sample with cooperation of an external pressure device. The top interface 2-6 is independently designed with pore pressure holes for test a pore pressure of the natural gas hydrate sample during triaxial test. The rubber cylinder 2-2 and the fixing ring 2-3 are combined to form a triaxial test reaction chamber. The triaxial inner wall 2-4 is filled with high-pressure seawater to retain the deep-sea high pressure, the triaxial outer wall 2-5 is provided with a water inlet and a water outlet, and circulating cold water is responsible for retaining the deep-sea low temperature.

A use method of the present disclosure is as follows:

After the triaxial test device for deep-sea coring retaining in-situ condition is carried by a research vessel to vicinity of a target area of the deep-sea energy soil reservoir, staff on the research vessel can obtain a deep-sea energy soil reservoir sample through a pressure-retaining drilling tool, and then the sample 1-3 is stored into an existing pressure-retaining transfer system. The natural gas hydrate pressure-retaining transfer system is used to cut and transfer the sample 1-3, and the sample 1-3 conforming to a length requirement of the triaxial test is stored in the sample transfer system 1. The ball valve 1-4 is closed to preserve a high-pressure environment of the samples 1-3. The sample transfer system 1 is in butt joint with the triaxial machine system 2 under the action of the hold hoop 1-5. The tube-removing piston 1-2 is used to remove the sample 1-3 from the tube and then send the sample into the triaxial machine system 2. The triaxial test is carried in the rubber cylinder 2-2, thus mechanical properties of the deep-sea energy soil reservoir sample are obtained.

The triaxial test device for deep-sea coring retaining in-situ condition suitable for a shipborne laboratory has the following characteristics:
1. The device adopts a specially designed tube-removing piston 1-2 structure. After obtaining the fidelity natural gas hydrate sample, the plastic tube attached the outer side of the sample 1-3 can be removed, and then the sample can be sent into the reaction chamber, which is convenient for the subsequent triaxial test of the sample. Moreover, the piston structure also serves as an axial pressure providing source of the triaxial test, which simplifies the operation process and improves the test efficiency.
2. The device adopts a specially designed axial triangular throttle groove structure, and the triangular throttle area of the buffer device gradually decreases with the increase of the buffer stroke, thus making the change of the buffer pressure evenly during the transfer of the sample 1-3 and reducing the impact pressure.
3. The device adopts a specially designed triaxial machine system 2 structure with an inner layer and an outer layer. The inner layer is filled with high-pressure seawater to retain deep-sea high pressure, the outer layer is provided with a water inlet/outlet, and the circulating cold water is responsible for retaining deep-sea low temperature, thus retaining the temperature and pressure of the sample during the triaxial test, and preventing adverse impact of hydrate decomposition on mechanical properties of a test sample.
4. The device adopts a specially designed structure with the rubber cylinder 2-2 and the fixing ring 2-3, and a certain strength can be retained to facilitate the transfer of the natural gas hydrate sample on a premise of ensuring the transfer of confining pressure required by triaxial test.
5. The triaxial test device for deep-sea coring retaining in-situ condition can be carried on the research vessel conveniently, which can in butt joint with an existing natural gas hydrate sample pressure-retaining transfer system, thus performing a fidelity triaxial test immediately after obtaining the natural gas hydrate sample, and achieving strong scientific research benefits.

It should be noted that it is apparent to those skilled in the art that the present disclosure is not limited to the details of the above exemplary embodiments, and can be realized in other specific forms without departing from the spirit or basic characteristics of the present disclosure. Therefore, the embodiments should be considered as exemplary and non-limiting in all aspects, and the scope of the present disclosure is defined by the appended claims rather than the above description, so it is intended to embrace all changes that fall within the meaning and range of equivalents of the claims, and any reference signs in the claims should not be regarded as limiting the claims involved.

Specific examples are used herein for illustration of the principles and embodiments of the present disclosure. The description of the embodiments is merely used to help illustrate the method and its core principles of the present disclosure. In addition, a person of ordinary skill in the art can make various modifications in terms of specific embodiments and scope of application in accordance with the teachings of the present disclosure. In conclusion, the content of this specification shall not be construed as a limitation to the present disclosure.

1. A triaxial test device for deep-sea coring retaining in-situ condition suitable for a shipborne laboratory, comprising:
a sample transfer system, configured to remove a natural gas hydrate sample from a tube and send the natural gas hydrate sample to a triaxial machine system, wherein the sample transfer system comprises:
a sample cylinder configured to receive the natural gas hydrate sample,
a tube-removing piston arranged in the sample cylinder and configured to push the sample to enter a triaxial machine system,
a ball valve, the sample cylinder being connected to the ball valve through bolts, and
a connector, and
a triaxial machine system, configured to implement a triaxial test of the natural gas hydrate sample in a vacuum condition, wherein the triaxial host machine system comprises:
a bottom interface hermetically connected to the sample cylinder through the connector,
a rubber cylinder in communication with the bottom interface,
a triaxial inner wall, the rubber cylinder being arranged in an inner cavity of the triaxial inner wall
a triaxial outer wall located at a periphery of the triaxial inner wall and having a water inlet and a water outlet, wherein circulating cold water communicates with the water inlet and the water outlet, and
a top interface;
wherein the sample is sent into the rubber cylinder for a triaxial compression test, and the triaxial inner wall is filled with seawater for guaranteeing a test pressure.

2. The triaxial test device for deep-sea coring retaining in-situ condition suitable for a shipborne laboratory according to claim 1, wherein the connector comprises a hold hoop, and the bottom interface is hermetically connected to the sample cylinder through the hold hoop.

3. The triaxial test device for deep-sea coring retaining in-situ condition suitable for a shipborne laboratory according to claim 1, wherein a tail end of the tube-removing piston is provided with a triangular groove symmetrical up and down, and when the sample is stored in the sample cylinder, oil between the tube-removing piston and the sample cylinder flows out through the triangular groove in an axial direction.

4. The triaxial test device for deep-sea coring retaining in-situ condition suitable for a shipborne laboratory according to claim 1, wherein a plastic tube is attached to an outer side of the sample placed in the sample cylinder, and a diameter of the tube-removing piston is smaller than an inner diameter of the plastic tube.

5. The triaxial test device for deep-sea coring retaining in-situ condition suitable for a shipborne laboratory according to claim 4, wherein a diameter of the bottom interface is smaller than an outer diameter of the plastic tube, the tube-removing piston in the sample transfer system is able to push the sample to move forwards under an action of seawater until the sample is abutted against the bottom interface of the triaxial machine system, and the sample is separated from the plastic tube under pushing of the tube-removing piston to enter the triaxial machine system.

6. The triaxial test device for deep-sea coring retaining in-situ condition suitable for a shipborne laboratory according to claim 1, wherein each of the bottom interface and the top interface is provided with confining pressure holes, and the confining pressure holes on the bottom interface and the top interface are configured to provide a confining pressure for the sample with cooperation of an external pressure device.

7. The triaxial test device for deep-sea coring retaining in-situ condition suitable for a shipborne laboratory according to claim 1, wherein the top interface is provided with pore pressure holes, the pore pressure hole is configured for testing a pore pressure of the sample during the triaxial test.

8. The triaxial test device for deep-sea coring retaining in-situ condition suitable for a shipborne laboratory according to claim 1, wherein the rubber cylinder is fixedly installed in an inner cavity of the triaxial inner wall through a fixing ring.
